(19) United States
(12) Patent Application Publication
(10) Pub. No.: US 2023/0293525 A1
(43) Pub. Date: Sep. 21, 2023

(54) METHODS AND COMPOSITIONS FOR THE TREATMENT OF VIRAL INFECTIONS

(71) Applicant: University of Connecticut, Farmington, CT (US)

(72) Inventor: Rahul N. Kanadia, Storrs, CT (US)

(21) Appl. No.: 18/006,235

(22) PCT Filed: Jul. 30, 2021
(86) PCT No.: PCT/US2021/043863

§ 371 Date: Jan. 20, 2023

Related U.S. Application Data
(60) Provisional application No. 63/058,864, filed on Jul. 30, 2020.

(51) Int. Cl. A61K 31/513; A61K 31/17; A61P 31/14; C12N 15/113

(52) U.S. Cl. CPC A61K 31/513; A61K 31/17; A61P 31/14; C12N 15/1131; C12N 2310/14

(57) ABSTRACT

A method inhibiting replication of an RNA/DNA virus in a viral host comprises contacting the viral host with a minor spliceosome inhibitor and inhibiting the replication of the RNA/DNA virus, wherein the RNA/DNA virus interacts with minor intron-containing genes (MIGs) in the viral host. Also included are methods of treating a subject having or suspected of having an RNA/DNA viral infection.

### CROSS-REFERENCE TO RELATED APPLICATIONS

This application claims priority to U.S. Provisional Application 63/058,864 filed on Jul. 30, 2020, which is incorporated herein by reference in its entirety.

### STATEMENT REGARDING FEDERALLY SPONSORED RESEARCH & DEVELOPMENT

This invention was made with government support under R01 NS102538-03 awarded by the National Institutes of Health. The government has certain rights in the invention.

### BACKGROUND

The pandemic caused by SARS-CoV-2 presents a clear and present danger, urgently requiring us to design new anti-viral strategies. As the ultimate defense against viruses, vaccines are under development but usually take more time to develop and may not provide complete immunity. Therefore, potent anti-viral therapeutics are needed to slow the progression of the spread of SARS-CoV-2 and other viruses,

### BRIEF SUMMARY

In one aspect, a method of inhibiting replication of an RNA/DNA virus in a viral host comprises contacting the viral host with a minor spliceosome inhibitor and inhibiting the replication of the RNA/DNA virus, wherein the RNA/DNA virus interacts with minor intron-containing genes (MIGs) in the viral host.

In another aspect, a method of treating a subject having or suspected of having an RNA/DNA viral infection, comprising administering to the subject a. minor spliceosome inhibitor, wherein the RNA/DNA virus interacts with minor intron-containing genes (MIGs) in a host.

### BRIEF DESCRIPTION OF THE DRAWINGS

FIG. 1 shows that MIGs interact with viral proteins across crucial stages of SARS-CoV-2 lifecycle. Schematized here is the life cycle of SARS-CoV2, starting with the Spike protein (S)-receptor mediated endocytosis and ending with successful SARS-CoV2 viral release. Here is shown viral proteins (black underlined) and their interacting MIGs (red) as reported in the art across key stages of the viral lifecycle. PRIM1 and POLA2 interact with viral NSP1 which function to block host translation and is also is crucial for the assembly of the Replication/Transcription Complex (RTC); SBNO1 and CRTC3 interact with viral NSP12, which is the RNA-Dependent RNA Polymerase (RdRP) enzyme of the RTC, and thus is essential for viral genome replication; AP2A2 interacts with NSP10 that forms a heterodimer with NSP16 and is essential for viral mRNA capping to evade host immune surveillance; EXOSC2, EXOSC5 and SRP72 interact with NSP8, the primase of the RTC; HSBP1 interacts with NSP13, which is a helicase that unwinds double stranded RNA and DNA in 5′ to 3′ direction; NUP2IO interacts with NSP4 which, with NSP3, is essential for viral replication, helping target the RTC; INTS4 and ATP6V1A interact with Membrane (M) protein, which along with Envelope (E), Nucleocapsid (N), and S proteins locate to the ER membrane in the host cells; UPF1 interacts with N which is located in the cytoplasm and binds to protects genomic viral RNA upon genomic replication.

FIGS. 2A-2E show enrichment of MIGs in viral targets and host factors. FIG. 2A shows less than 0.5% of the introns in human genes are U12-type or minor introns that are spliced by the minor spliceosome. In FIG. 2B, it was observed thatMIGs exhibited stronger enrichment in sets of human proteins that viruses target through interactions between viral and human proteins in comparison to alternatively spliced isoforms (P<10⁻¹⁰, Student's t-test), when a variety of different viruses were focused on. FIG. 2C shows MIGs are enriched in sets of host factor genes that are required by different viruses to infect their host cells (Error bars indicate 95% confidence interval, P<10⁻¹⁰, Student's t-test). FIG. 2D shows that in a network of human protein-protein interactions, MIGs were preferably enriched in bins of highly interacting proteins. Notably, we found an enforced trend when we considered MIGs in the essentialome. FIG. 2E shows that in utilizing 220 MIGs that were targeted by diverse viruses, it was observed that, overall, such genes hardly changed in their expression values (log2FC) in samples from lung biopsies of SARS-CoV-2 patients, and MRCS cell lines 24 h post-infection with MERS-CoV and SARS-CoV-1.

FIGS. 3A-3F show the efficacy of inhibiting the minor spliceosome. FIG. 3A shows MIGs were enriched in a set of gene targets of drugs that appeared promising in treating SARS-CoV-2 infection, The enrichment signal was amplified when we only considered MIGs that interacted with gene targets of SARS-CoV-2 in a network of human protein-protein interactions Error bars indicate 95% confidence interval, P-values refer to results obtained with Fisher's exact test). FIG. 3B) shows that usually, only one intron in MIGs is a minor intron that is spliced through the minor spliceosome. Disruption of the minor spliceosome results in retention of the minor intron and/or alternative splicing across minor intron through the major spliceosome, generating transcripts targeted for nonsense mediated decay (NMD) or aberrant proteins. Focusing on different snRNA components of the minor spliceosome we achieved its inhibition genetically by ablating the U11 snRNA in the developing cortex of mice, mutations in the U4atac snRNA of human peripheral blood mononuclear cells (PBMC) and anti-sense morpholines against U12, U4atac and U6atac in human HEK-293t cells. In FIG. 3C, the Venn diagram indicates the overlaps of virus-interacting MIGs whose splicing is significantly affected through different ways of inhibiting the minor spliceosome. FIG. 3D shows that the SARS-CoV-2 targeted MIGs showed either elevated minor intron retention or elevated retention and alternative splicing upon minor spliceosome inhibition. In FIG. 2E, the heatmap with mis-splicing indices (MSI) reflect the level of minor intron retention in MIGs involved in SARS-CoV-2 life cycle upon minor spliceosome inhibition. FIG. 2F shows Sashimi plots that indicate elevated levels of alternative splicing around the minor intron ref MIGs GOLGA7 and ZDHHC5 in patients with mutations in the minor spliceosome-specific U4atac snRNA.

FIG. 4 shows a schematic of snRNA transcription regulation through P53-Mir-34 pathway (modified from Takahashi et al., Nat. Conimu. 2015, 6:5941).

FIG. 5 shows the regulation of U11 and U12 snRNA transcription by P53 (modified from Anwar et al., Biochimica, et hiophysica acta, 2016, 1859 (8) 975-982.

FIG. 6 shows upregulation of U11, U4atac and U12 snRNA (modified from Samuel et al., Oncotraget, 2016, vol 7 (31): 49611-22.

FIG. 7 shows elevated minor intron retention in cold exposed tilapia hypothalamus. Boxplot showing 5^{th}-95^{th} percentile of minor intron retention levels in control and cold exposed Nile tilapia. Significance was determined by Mann-Whitney U-test. ***=P<0.001.

FIG. 8 shows that that treating HEK293 cells with Tenovin 1 successfully downregulates the crucial minor spliceosome snRNAs including U11, U12, U6atac, and U4atac.

FIG. 9 shows Tenovin1 inhibits replication of Huh7 cells infected with Ebola virus expressing GFT imaged in the morning.

FIG. 10 shows Tenovin1 inhibits replication of VeroE6 cells infected with Ebola virus expressing GFP imaged in the morning.

The above-described and other features will he appreciated and understood by those skilled in the art from the following detailed description, drawings, and appended

### DETAILED DESCRIPTION

In general, viruses have co-evolved with their hosts over millions of years, shedding genes that are necessary for their life cycle in favor of a compact genome (G. L Kajan, A. Doszpoly, Z. L. Tarjan, M. Z. Vidovszky, T. Papp, Virus-Host Coevolution with a Focus on Animal and Human DNA Viruses, Journal of molecular evolution 88, 41 (Jan, 2020)), To recover lost functions, proteins of SARS-CoV-2, including non-structural (Nsp) and structural proteins (nucleocapsid-N, spike-S, membrane-M, envelop-E), interact with human host proteins to replicate the genome and package new virus particles. Mutations underpinning the emergence of new viral strains such as SARS-CoV-2 do not fundamentally alter the life cycle of the virus, suggesting that virus-host PPIs can be leveraged to design antiviral drugs. Indeed, out of 332 host proteins that were recently shown to interact with various SARS-CoV-2 proteins, 62 were reported drug targets.

Many of the host proteins that viruses interact with are minor intron-containing genes (MIGs). Minor introns, which can only be spliced by the minor spliceosome, are unique introns that are found in a small number (<2%) of genes. Since proper expression of MIGs requires the minor spliceosome, the minor spliceosome can be leveraged to disrupt the expression of MIGs. Inhibition of the minor spliceosome, will disrupt expression (e.g., transcription, splicing, mRNA transport, mRNA turnover and/or protein production) of MIGs that are used by viruses for the propagation of their life cycle. Therefore, drugs and methods that inhibit the minor spliceosome can act as antiviral drug/strategy against SARS-CoV-2. Herein, blocking the virus life cycle by disrupting the host proteins that the viruses need to propagate their life cycle is described. Since MiGs are highly enriched in the list of host genes that the viruses use for their life cycle, it is proposed that by disrupting MIGs viruses need through inhibiting the minor spliceosome.

The minor spliceosome is a ribonuclear protein (RNP) complex that splices minor introns in the human genome. It includes four unique small nuclear RNAs (snRNAs), U11, U12, U4atac and U6atac snRNA, and unique proteins including, U11/U12-65K, U11/U12-59K, SNRNP48, SNRPN35, SNRNP31, SNRNP25, SNRNP20.

In an aspect, in a viral host comprises contacting the viral host with a minor spliceosome inhibitor and inhibiting the replication of the RNA/DNA virus, wherein the RNA/DNA virus interacts with minor intron-containing genes (Mikis) in the viral host.

Exemplary RNA/DNA viruses include SARS-CoV-2, SARS-CoV1, MERS-CoV, Hepatitis C (RNA virus), Epstein-Barr virus (dsDNA virus), HPV (DNA virus), Ebola (RNA virus), HIV-1 (RNA virus), Herpes virus (DNA virus), Influenza A (RNA virus), and Zika (RNA virus).

Exemplary spliceosome inhibitors include inhibitory nucleic acid against minor spliceosome components, such as an siRNA, morpholino or an antisense oligonucleotide, or a small molecule.

In an aspect, the minor spliceosome inhibitor targets an RNA selected from U11, U12, U4atac and U6atac snRNA; or a protein selected from RNPC3, PDCD7, SNRNP48, SNRNP35, ZCRB1 (SNRNP31), SNRNP25, ZMAT5 (SNRNP20). In an aspect, the minor spliceosome inhibitor is an siRNA which inhibits the synthesis of RNPC3, PDCD7, SNRNP48, SNRNP35, ZCRB1 (SNRNP31), SNRNP25 or ZMAT5 (SNRNP20).

In an aspect, anti-sense oligonucleotides, morpholinos, and siRNAs that bind the U12, U4atac and U6atac snRNAs can be used to inhibit the minor spliceosome.

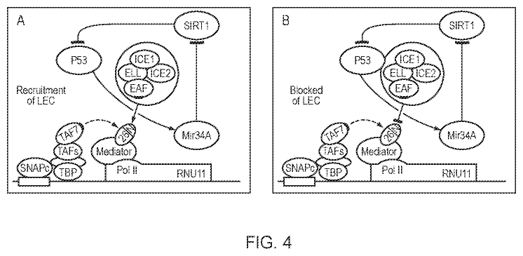

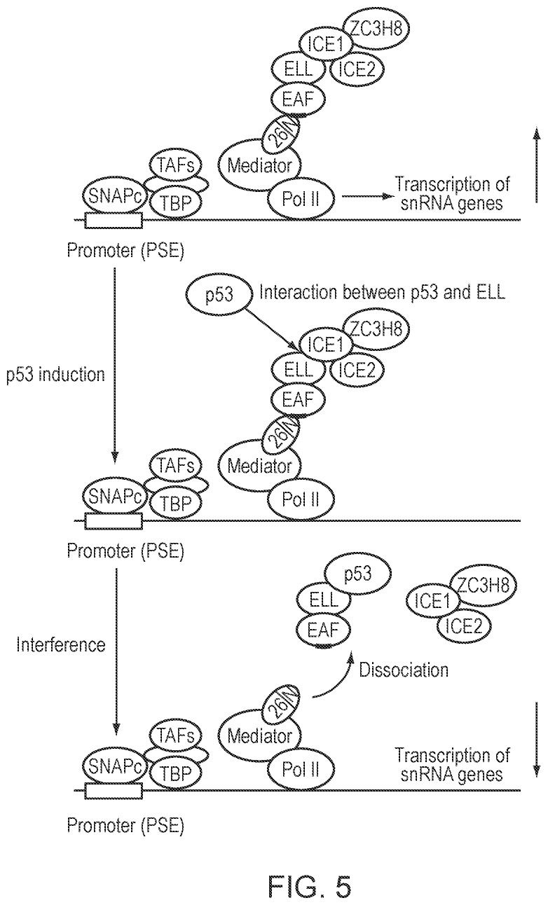

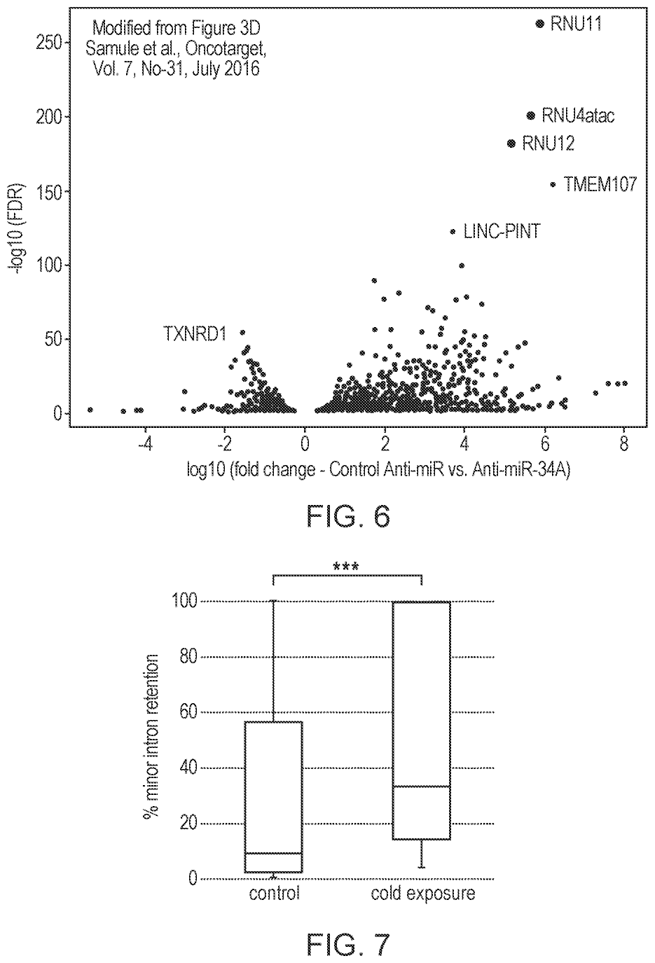

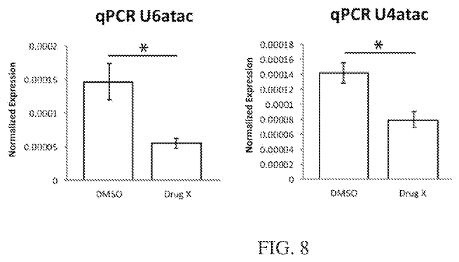

Alternatively, drugs that upregulate P53 can be used, as downregulation of P53 alongside inhibition of Mir-34A results in upregulation of Rnu11 and Rnu12 (FIG. 4). FIG. 5 shows the regulation of U11 and U12 snRNA transcription by P53. For example, Sirt1 and Sirt2 normally downregulate P53, therefore drugs such as Tenovin 1 and 6 that downregulate Sirt1 and Sirt2 will indirectly upregulate P53. Upregulation of P53 in turn will downregulate the expression of U11 and U12 snRNAs, which will ultimately result in the inhibition of the minor spliceosome.

More specifically, the rationale is that P53 normally disrupts the formation of little elongation compex (LEC), which is required for the transcription of U11 and U12 snRNA. Therefore, P53 is a regulator of transcription of U11 and U12 snRNAs. Thus, increasing levels of P53 will increase levels of U11 and U12 snRNA. One way to increase levels of P53 is to inhibit those factors that generally reduce the levels of P53. One such factor is SIRT1, which is shown to downregulate P53 levels. Therefore, by inhibiting Sirt1, we can relieve the transcriptional inhibition of P53 and ultimately upregulate the levels of P53, which in turn will reduce the levels of U11 and U12 snRNA. Another strategy is to increase the levels of Mir34A, which is a downstream effector of P53, and targets Sirt1 mRNA thereby reducing the levels of Sirt1. Consequently, the levels of P53 will be upregulated and U11 and U12 snRNA levels will be downregulated, which will ultimately result in the inhibition of the minor spliceosome.

Another downstream target of P53 is mir34A, which downregulates Sirt1 and Sirt2. FIG. 6 shows upregulation of U11, U4atac, and U12 snRNA. P53-null primary fibroblasts treated with anti-Mir34A oligo reveal upregulation of U11, U12 and U4atac snRNA that is represented in a volcano plot. Overexpression of mir34A should downregulate Sirt1 and Sirt2 and in turn upregulate P53, which through the downregulation of U11 and U12 snRNA will inhibit the minor spliceosome, in an aspect, the minor spliceosome inhibitor is mir34A.

In another aspect, the inventors have discovered that cold-stress in fish inhibits the minor spliceosome, which can be detected through elevated minor introns from RNAseq performed on the dissected hypothalamus. FIG. 7 shows elevated minor intron retention in cold exposed tilapia hypothalamus. Therefore, another way to inhibit the minor spliceosome is to induce intermittent hypothermia, which will inhibit the minor spliceosome and block the virus. While hypothermia is a not an ideal therapeutic strategy, we could “trick” the cells into thinking that they are experiencing hypothermia by activating the cold or menthol receptor called TRPM8. Here Icilin, which is known to selectively activate TRPM8 channel, can be used to inhibit the minor spliceosome. In an aspect, the minor spliceosome inhibitor is an activator of the TRPM8 receptor, such as Icilin.

In another aspect, the RNA virus is SARS-CoV-2 and the MIGs include PRIM1, POLA2, SBNO1, CRTC3, AP2A2, EXOSC2, EXOSCS, SRP72, HSBP1, NUP210, INTS4, ATP6V1A, UPF1, or a combination thereof.

Also included herein are pharmaceutical compositions comprising a minor spliceosome inhibitor and a pharmaceutically acceptable excipients such as diluents, preservatives, soluhilizers, emulsifiers, and adjuvants. As used herein “pharmaceutically acceptable excipients” are well known to those skilled in the art.

Tablets and capsules for oral administration may be in unit dose form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol. or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs; or may he presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan nionooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxyhenzoate or sorbic acid, and if desired conventional flavoring or coloring agents.

The active ingredient may also be administered parenterally in a sterile medium, either subcutaneously, or intravenously, or intramuscularly, or intrasternally, or by infusion techniques, in the form of sterile injectable aqueous or oleaginous suspensions. Depending on the vehicle and concentration used, the drug can either be suspended or dissolved in the vehicle. Advantageously, adjuvants such as a local anaesthetic, preservative and buffering agents can he dissolved in the vehicle.

Pharmaceutical compositions may conveniently be presented in unit dosage form and may be prepared by any of the methods well known in the art of pharmacy. The term “unit dosage” or “unit dose” means a predetermined amount of the active ingredient sufficient to be effective for treating an indicated activity or condition. Making each type of pharmaceutical composition includes the step of bringing the active compound into association with a carrier and one or more optional accessory ingredients. In general, the formulations are prepared by uniformly and intimately bringing the active compound into association with a liquid or solid carrier and then, if necessary, shaping the product into the desired unit dosage form.

In a further aspect, a method of treating a subject having or suspected of having an RNA/DNA viral infection comprises administering to the subject a minor spliceosome inhibitor, wherein the RNA/DNA virus interacts with minor intron-containing genes (MIGs) in a host. Exemplary subjects are mammalian subject, specifically human subjects.

In an aspect, the minor spliceosome inhibitor is administered for short-term therapy. Here short-term therapy would be the window of time when the viral load needs to he reduced so that a patient can be provided with other therapies for the symptoms that remain from the original infection. Thus, the minor spliceosome inhibitor could be administered until the virus is eliminated and no longer detected in the patient.

The invention is further illustrated by the following non-limiting examples.

### EXAMPLES

### Example 1

### Identification of IGFs in Viruses

### Methods

Determination of the essentialome. Meyers et al. identified genes that are essential for the survival of 342 rapidly dividing cancer cell lines correcting for gene copy numbers per cell line, employing a genomewide CRISPR/Cas9 screen. In accordance with recommendations from the Broad Institute, we removed the PK59_PANCREAS cell line from our analysis, since it had failed subsequent quality controls. Classification of the remaining 341 cell lines by cancer origin/type was performed based on the cell line data. Based on their thresholding we identified 4,360 genes in the essentialome.

Alternative splicing genes. 13,595 genes were collected that have alternative spliced isoforms from the VastDB database.

Enrichment analysis. Considering proteins with a certain characteristic d (e.g., being a certain distance away from a reference protein), we calculated the fraction of proteins that had a feature i in each group d, fᵢ(d), As a null model, we randomly sampled protein sets with feature i of the same size 10,000 times and calculated the corresponding random fraction, f_{i,r}(d). The enrichment/depletion of proteins with feature i in a group d was then defined as Eᵢ(d)=1g₂[fᵢ(d)/f_{l,r}(d)].

Protein-protein interactions. As for a set of high-quality human protein-protein interactions we utilized 165,058 interactions between 15,619 human proteins as of the HINT database.

Human-virus protein interactions. Collecting data from the HPIDB database, we used 916 human proteins that were targeted by the Hepatitis C virus, as well as 856 targets of the Herpes simplex virus, 447 targets of HIV-1, 1,320 targets of Influenza A virus, 144 targets of the Ebola virus, 964 targets of the Epstein-Barr virus, 374 targets of the Zika virus and 940 targets of the Human papilloma virus. As for SARS-Cov-2, 332 targets were collected from recent high-throughput screen.

Host factors. 262 genes were used that were required by Hepatitis C virus to infect a human host cell. For Herpes simplex virus, 358 such genes were collected. Furthermore, 917 such genes of HIV-1, 1,251 genes of Influenza A virus, 315 proteins of the Human papilloma virus, 144 genes of the Epstein-Barr-virus, 790 proteins of the Zika virus and 237 proteins of the Ebola virus were utilized.

Promising SARS-CoV-2 drugs. In a set of 87 drugs that were screened for SARS-CoV-2 viral inhibition from Table S2 in we collected 46 drugs that led to a ratio of CC50 (cell viability) to IC50 (viral inhibition)≥2 were retained as promising drugs. Additional 11 promising drugs were obtained based on their shared values for cell viability and virus growth. Such a set of drug targets translated to a total of 135 drug targets.

Gene expression. Data from samples infected with three different coronaviruses, including SARS-CoV-2 (GSE150316), MERS-CoV and SARS-CoV-1 (GSE56192), were analyzed for changes in gene expression. Raw counts from RNAseq data pertaining to lung biopsies of SARS-CoV-2 patients and healthy controls was downloaded from SRA. DESeq2 was utilized to process the data, applying variance stabilizing transformation (VST), and to calculate differential expression. Outliers were removed based on principal component analysis (PCA). Log2 fold changes and adjusted p-values were retrieved for those minor intron containing genes (MIGs) within the SARS-CoV-2 interactome to determine whether any of the MIGs were differentially expressed. Additionally, DESeq2-processed data was downloaded for MERS-CoV and SARS-CoV-1. Data pertaining to MRCS cell lines 24 h post-infection at a multiplicity of infection (MOI) of 3 was included in the current analysis. Log2 fold changes and adjusted p-values were retrieved for MIGs targeted by numerous viruses. The pheatmap R package was used to generate a heatmap of the gene expression of these MIGs across all of the datasets.

Minor intron retention and alternative splicing around minor introns. Retention and alternative splicing of minor introns was evaluated in three RNAseq datasets where the minor spliceosome was inhibited (GSE96616). Reads were aligned to the mm10 or hg38 genome using Hisat2, followed by extraction of the uniquely mapped reads. The level of minor intron retention and alternative splicing around minor introns was then reported as a mis-splicing index (MSI) as described previously. Briefly, for minor intron retention, exon-minor intron boundary reads were quantified using BEDTools and normalized to the number of spliced reads mapping to the canonical exon-exon junction. For alternative spliced reads supporting alternative junctions around the minor intron were quantified and normalized to the number of spliced reads mapping to the canonical exon-exon junction. Significant differences in MSI values between control and minor spliceosome-inhibited samples were determined using a Student's t-test cKO) or one-way ANOVA (niorpholinos). Since no statistical analyses could be performed for patient data (n=1), elevated retention or alternative splicing was determined as a >2-fold increase in MS1 value.

Open reading frame analysis. The effect of retention and/or alternative splicing on the open reading frame of MIGs was determined using ExPASy. To determine whether a premature stop codon would activate NMD, the distance of the premature stop codon to a downstream exon-junction complex was evaluated. A distance of >50 nt is predicted to activate the NMD pathway, whereas in other instances it would produce a protein. The effect of the alternative splicing on protein domains was determined by blasting the aberrant protein sequences on the pfam database.

### Results

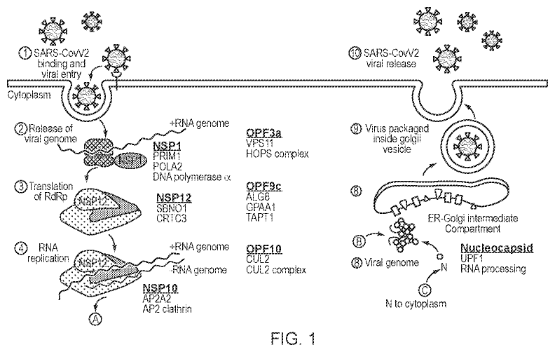

The pandemic caused by the severe acute respiratory syndrome coronavirus 2. (SARS-CoV-2) presents a clear and present danger, urgently requiring us to design new anti-viral strategies. As the ultimate defense against viruses, vaccines are under development, hut usually take more time to develop and may not provide complete immunity. Therefore, potent anti-viral therapeutics are needed to slow the progression of the spread of SARS-CoV-2. In general, viruses have co-evolved with their hosts over millions of years, shedding genes that are necessary for their life cycle in favor of a compact genome. To recover lost functions, proteins of SARS-CoV-2, including non-structural (lisp) and structural proteins (nucleocapsid-N, spike-S, membrane-M, envelop-E), interact with human host proteins to replicate the, genome and package new virus particles. Mutations underpinning the emergence of new viral strains such as SARS-CoV-2 do not fundamentally alter the life cycle of the virus, suggesting that virus-host PPIs can be leveraged to design antiviral drugs. Indeed, out of 332 host proteins that were recently shown to interact with various SARS-CoV-2 proteins, 62 were reported drug targets. Utilizing such a map of host-virus protein interactions, we surprisingly observed that SARS-CoV-2 interacts with 20 minor intron containing genes (MIGs) in almost every stage of the viral life cycle (FIG. 1).

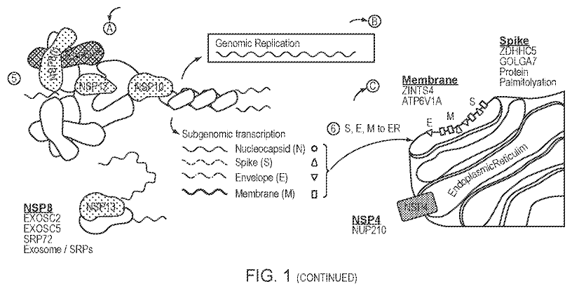

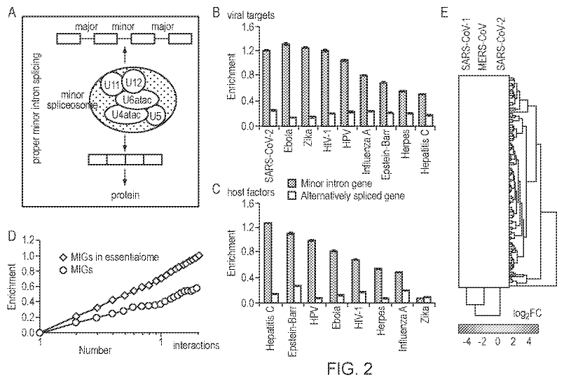

Utilizing all 699 identified human MIGs, we observed that they are significantly enriched in the human-SARS-CoV-2 interactome (P<5×10⁻⁴, Fisher's exact test), In particular, <0.5% of the introns are U12-type or minor introns that are spliced by the minor spliceosome (5, 6), while the rest, U2-type or major introns, are spliced by the major spliceosome (FIG. 2A). As MIGs usually also possess several major introns, disruption of the minor spliceosome results in minor intron retention and/or major spliceosome-mediated alternative splicing across minor introns that usually leads to degradation of the transcript through nonsense mediated decay (NMD) or the production of aberrant proteins. Next, it was hypothesized that this finding generalizes to other viruses. Indeed, significant enrichment of MIGs in interactions between the human host and proteins of Zika, HIV-1, HPV; Influenza A, Epstein-Barr, Ebola, Herpes and Hepatitis C (FIG. 2B) was observed. Additionally, the same type of analysis was carried out using genes with alternatively spliced isoforms, a set of genes with features that, similar to minor introns, are inherent to gene structure. Compared to genes with alternatively spliced isoforms it was found that enrichment of MIGs in all different viral-host interactomes was stronger and statistically significant (P<10⁻¹⁰), Student's test). Anticipating that MIGs in general are required by viruses to replicate, we also considered host factors, defined as sets of genes that, when inhibited, halted the viral life cycle. Similar to the viral-host interactomes, MIGs were significantly enriched in host factors of Zika, 1-IPV, Influenza A, Epstein-Barr, Ebola, Herpes and Hepatitis C (FIG. 2C). As in the previous analysis, enrichment of MiGs was notably stronger compared to alternatively spliced genes (P<10⁻¹⁰, Student's test), supporting the notion that viruses have evolved to leverage the functions of this crucial set of MIGs.

Viruses typically target host proteins, i.e. hubs, as they are involved in a high number of interactions with other host proteins. Generally, such hubs are essential for survival and evolutionarily conserved. Hypothesizing that MIGs are putative hubs in the human PPI network we indeed found that they were significantly enriched in bins of highly interacting proteins (FIG. 2D). Previously, it was shown that MIGs are also significantly enriched in the essentialome, a set of highly conserved genes essential for survival, going back to the last eukaryotic common ancestor (LECA). Assuming that viruses utilize functions of MIGs that remain relatively unchanged across evolution, we found that the enrichment of MIGs in the essentialome was further enhanced in bins of highly interacting proteins (FIG. 2D). As a corollary, it was hypothesized that the expression of MIGs remains largely unaltered post virus infection, providing a stable gateway to tap the host cellular machinery. Thus, the transcriptomic response of samples infected with SARS-CoV-2, MERS-CoV (Middle East respiratory syndrome-related coronavirus) and SARS-CoV-1 was examined. Indeed, the heatmap in FIG. 1E indicated that those 220 MIGs identified in all virus-host interactomes were mostly unchanged post coronavirus infection. Specifically, it was observed that none of the MIGs in the SARS-CoV-2 host interactome were differentially expressed.

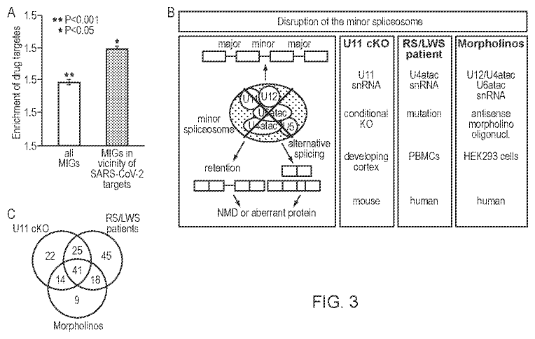

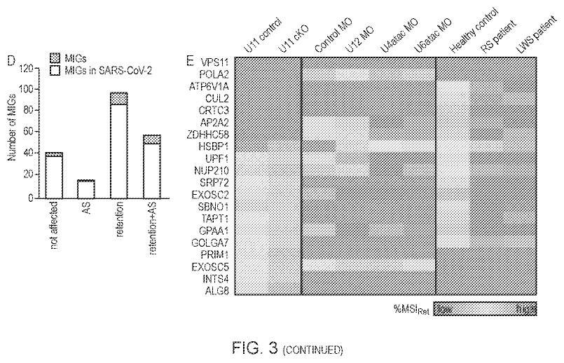

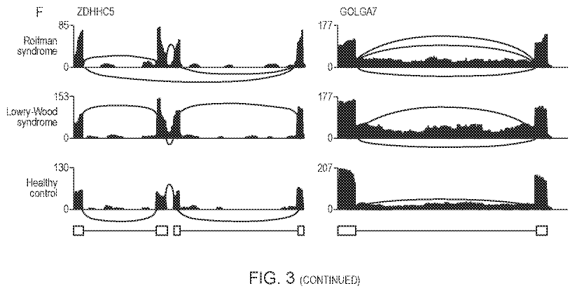

All of these findings point to the notion that MIGs encode host proteins critical for the virus life cycle, suggesting that the disruption of MIGs could potentially block viral replication. In particular, a set of 57 drugs were collected that showed promise in treating cells that were infected with SARS-CoV-2. Remarkably, we observed that MIGs were significantly enriched in the corresponding set of 135 genes that are drug targets (FIG. 3A). Such a trend was significantly amplified when we focused on MIGs that interacted with gene targets of SARS-CoV-2 (Suppl. Materials) (P<0.05, Fisher's exact test).

While MIGs could be targeted separately through individual drugs, their Simultaneous disruption could block disparate aspects of the virus life cycle at the same time, pointing to an efficient, ideal anti-viral therapeutic strategy. Previously, the transcriptomic response to minor spliceosome inhibition in a number of systems, by targeting different components within this machinery (FIG. 3B) was analyzed. In particular, the minor spliceosome was inhibited by genetically ablating the U11 snRNA in the developing cortex (Rnu11^{Flx/Flx}::Emx1-Cre⁺) and limb, and used anti--sense morpholinos against U12, U4atac and U6atac HEK-293t cells. Moreover, human mutations in RNU4ATAC result in the inhibition of the minor spliceosome and lead to Roffman syndrome and Lowry-Wood syndrome. Notably, it was observed that the sets of MIGs that were affected by the inhibition of the minor spliceosome largely overlapped (FIG. 3C).

Subsequently, it was determined whether the 220 MIGs that were involved in virus-host interactomes were mis-spliced. Strikingly, it was found that 174 (79.1%) of these MIGs showed either significantly up-regulated retention and/or alternative splicing across minor introns in one or more of these minor spliceosome inhibition datasets (FIG. 3D). Moreover, functional annotation analysis of the 174 MIGs through DAVID showed significant enrichment of Gene Ontology (GO) terms that pertain to viral process, poly(A) RNA binding, Ran-GTPase binding, intracellular protein transport, protein import into nucleus-docking, late endosome and others, suggesting that MIGs participate in crucial cellular processes that are leveraged by the virus for its life cycle. Focusing on MIGs that are targeted by SARS-CoV-2, it was found that almost all MIGs showed either elevated minor intron retention and/or elevated alternative splicing upon minor spliceosome inhibition (FIG. 3E). For such MIGs, minor intron retention resulted in a frameshift and the introduction of a premature stop codon. All of these premature stop codons were located >50 nt upstream of an exon-junction complex, and therefore predicted to activate nonsense-mediated decay (NMD), resulting in degradation of the aberrant MIG transcripts. In contrast, alternative splicing across the minor intron, which was observed in the MIGs ZDHLIC5, SRP72, EXOSC2, SBNO1, TAPT1, GOLGA7 and ALG8, was predicted to result in the production of truncated proteins for 5 out of 7 MIGs. Importantly, inhibition of the minor spliceosome would result in aberrant alternative splicing of GOLGA7 and ZDHHC5, two MIGs that are crucial for post-translation processing and incorporation of the Spike protein of SARS-CoV-2 into the membrane (FIG. 3F).

In all, it was shown that MIGs are ancestral, essential genes that many viruses significantly leverage to propagate their life cycle. These analyses and data strongly support that individual MIGs can be ideal antiviral targets; however, also presented herein are mechanisms to inhibit the minor spliceosome as a whole that would also yield the desired goal of discovering a novel and potent antiviral strategy. Thus, MIGs, in general, and the minor spliceosome, in particular, are introduced as novel players in our efforts to finding antiviral strategies against SARS-CoV-2 and other viruses.

TABLE 1
Identified MIGs

| | | Number | Benjamini |
|---|---|---|---|
| | | of | adjusted |
| GO-term | Description | genes | P-value |
| GO:0005654 | Nucleoplasm | 72 | 1.04E−14 |
| GO:0005515 | Protein binding | 133 | 7.21E−12 |
| GO:0005829 | Cytosol | 68 | 6.84E−09 |
| GO:0016032 | Viral process | 20 | 1.09E−07 |
| GO:0044822 | Poly(A) RNA binding | 34 | 1.36E−06 |
| GO:0016020 | Membrane | 47 | 6.32E−06 |
| GO:0008536 | Ran GTPase binding | 7 | 3.52E−05 |
| GO:0005730 | Nucleolus | 25 | 9.46E−05 |
| GO:0006886 | Intracellular protein transport | 14 | 2.76E−04 |
| GO:0008565 | Protein transporter activity | 8 | 4.37E−04 |
| GO:0034399 | Nuclear periphery | 5 | 0.001842 |
| GO:0005737 | Cytoplasm | 74 | 0.002101 |
| GO:0005643 | Nuclear pore | 7 | 0.002291 |
| GO:0004198 | Calcium-dependent cysteine-type | 5 | 0.002536 |
| | endopeptidase activity | | |
| GO:0005770 | Late endosome | 8 | 0.005211 |
| GO:0005769 | Early endosome | 10 | 0.009487 |
| GO:0000176 | Nuclear exosome (RNase complex) | 4 | 0.009578 |
| GO:0004532 | Exoribonuclease activity | 4 | 0.010585 |
| GO:0000178 | Exosome (RNase complex) | 4 | 0.010638 |
| GO:0005524 | ATP binding | 30 | 0.011204 |
| GO:0003723 | RNA binding | 16 | 0.011706 |
| GO:0000059 | Protein import into nucleus, | 4 | 0.01523 |
| | docking | | |
| GO:1900034 | Regulation of cellular response | 7 | 0.021388 |
| | to heat | | |
| GO:0043928 | Exonucleolytic nuclear-transcribed | 5 | 0.031741 |
| | mRNA catabolic process involved | | |
| | in deadenylation-dependent decay | | |
| GO:0043488 | Regulation of mRNA stability | 7 | 0.047828 |

### Example 2

### Downregulation of the Crucial Minor Spliceosome snRNAs including U11, U12, U6atac, and U4atac by Tenovin 1

Tenovin 1 was administered at 10 μM to HEK 293 cells in the presence of DMSO. Cells were incubated for 24 hours and the qPCR data was normalized to RN7SK. treating HEK293 cells with Tenovin1 successfully downregulates the crucial minor spliceosome snRNAs including U11, U12, U6atac, and U4atac. The reduction of these snRNAs should result in the inhibition of the minor spliceosome as these are crucial factors of the minor spliceosome. Therefore, we can use test whether it can block virus replication.

### Example 3

### Inhibition of Viral Replication

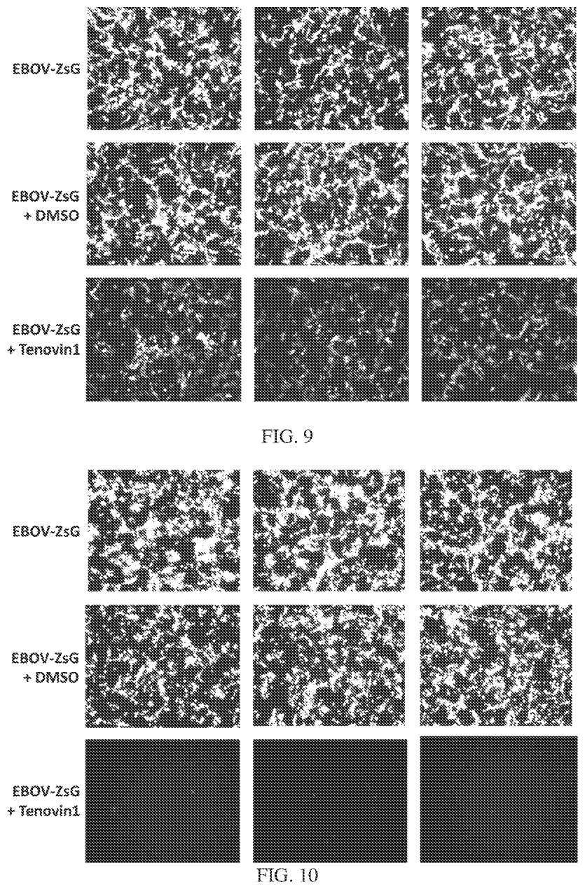

FIG. 9 shows Huh; cells were treated with either Tenovin1 or DMSO 24 hours prior to infecting the cells with Ebola virus expressing GFP. The morning image here reflect the reduction in the level of GFP treated with Tenovin1, which suggests that the virus is not replicating as fast as the sample with or without DMSO. Similar results were seen in the afternoon (data not shown). As shown in FIG. 10, similar results were observed in Vero6 cells infected with Ebola virus.

The use of the terms “a” and “an” and “the” and similar referents (especially in the context of the following claims) are to be construed to cover both the singular and the plural, unless otherwise indicated herein or clearly contradicted by context. The terms first, second etc. as used herein are not meant to denote any particular ordering, but simply for convenience to denote a plurality of, for example, layers. The terms “comprising”, “having”, “including”, and “containing” are to be construed as open-ended terms (i.e., meaning “including, but not limited to”) unless otherwise noted. “About” or “approximately” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±10% or 5% of the stated value. Recitation of ranges of values are merely intended to serve as a shorthand method of referring individually to each separate value failing within the range, unless otherwise indicated herein, and each separate value is incorporated into the specification as if it were individually recited herein. The endpoints of all ranges are included within the range and independently combinable. All methods described herein can he performed in a suitable order unless otherwise indicated herein or otherwise clearly contradicted by context. The use of any and all examples, or exemplary language (e.g., “such as”), is intended merely to better illustrate the invention and does not pose a limitation on the scope of the invention unless otherwise claimed. No language in the specification should be construed as indicating any non-claimed element as essential to the practice of the invention as used herein.

While the invention has been described with reference to an exemplary embodiment, it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment disclosed as the best mode contemplated for carrying out this invention, but that the invention will include all embodiments falling within the scope of the appended claims. Any combination of the above-described elements in all possible variations thereof is encompassed by the invention unless otherwise indicated herein or otherwise clearly contradicted by context.

1. A method of inhibiting replication of an RNA/DNA virus in a viral host, comprising contacting the viral host with a minor spliceosome inhibitor and inhibiting the replication of the RNA/DNA virus, wherein the RNA/DNA virus interacts with minor intron-containing genes (MIGs) in the viral host.

2. The method of claim 1, wherein the RNA/DNA virus comprises SARS-CoV-2, Hepatitis C (RNA virus), Epstein-Barr virus (dsDNA virus), HPV (DNA virus), Ebola (RNA virus), HIV-1 (RNA virus), Herpes virus (DNA virus), Influenza A (RNA virus), or Zika (RNA virus).

3. (canceled)

4. The method of claim 1, wherein a minor spliceosome inhibitor is an inhibitory nucleic acid selected from the group consisting of an siRNA, an antisense morpholino, and an antisense oligonucleotide.

5. The method of claim 4, wherein the minor spliceosome inhibitor targets an RNA selected from U11, U12, U4atac, and U6atac snRNA; or a protein selected from RNPC3, PDCD7, SNRNP48, SNRNP35, ZCRB1 (SNRNP31), SNRNP25, and ZMAT5 (SNRNP20).

6. The method of claim 4, wherein the minor spliceosome inhibitor is an siRNA that inhibits the synthesis of RNPC3, PDCD7, SNRNP48, SNRNP35, ZCRB1 (SNRNP31), SNRNP25 or ZMAT5 (SNRNP20) or an antisense oligonucleotide, antisense morpholino, or siRNA that binds U12, U4atac or U6atac snRNA.

7. (canceled)

8. The method of claim 1, wherein the minor spliceosome inhibitor is a drug that upregulates P53 or an activator of the TRPM8 receptor.

9. The method of claim 8, wherein the drug is Tenovin-1, Tenovin-6 or Icilin.

10. The method of claim 1, wherein the minor spliceosome inhibitor is mir34A.

11. (canceled)

12. The method of claim 1, wherein the RNA virus is SARS-CoV- 2 and the MIGs include PRIM1, POLA2, SBNO1, CRTC3, AP2A2, EXOSC2, EXOSCS, SRP72, HSBP1, NUP210, INTS4, ATP6V1A, UPF1, or a combination thereof.

13. A method of treating a subject having or suspected of having an RNA/DNA viral infection, comprising administering to the subject a minor spliceosome inhibitor, wherein the RNA/DNA virus interacts with minor intron-containing genes (MIGs) in a host.

14. The method of claim 13, wherein the minor spliceosome inhibitor is administered for short-term therapy until the virus is eliminated and no longer detected in the patient.

15. The method of claim 13, wherein the RNA/DNA virus comprises SARS- CoV-2, Hepatitis C (RNA virus), Epstein-Barr virus (dsDNA virus), HPV (DNA virus), Ebola (RNA virus), HIV-1 (RNA virus), Herpes virus (DNA virus), Influenza A (RNA virus), or Zika (RNA virus).

16. (canceled)

17. The method of claim 13, wherein the inhibitory nucleic acid is an siRNA, an antisense morpholino, or an antisense oligonucleotide.

18. The method of claim 17, wherein the minor spliceosome inhibitor targets an RNA selected from U11, U12, U4atac, and U6atac snRNA; or a protein selected from RNPC3, PDCD7, SNRNP48, SNRNP35, ZCRB1 (SNRNP31), SNRNP25, and ZMAT5 (SNRNP20).

19. The method of claim 13, wherein the minor spliceosome inhibitor is an siRNA which inhibits the synthesis of RNPC3, PDCD7, SNRNP48, SNRNP35, ZCRB1 (SNRNP31), SNRNP25 or ZMAT5 (SNRNP20) or an antisense oligonucleotide, antisense morpholino or siRNA that binds U12, U4atac or U6atac snRNA.

20. (canceled)

21. The method of claim 13, wherein the minor spliceosome inhibitor is a drug that upregulates P53 or an activator of the TRPM8 receptor.

22. The method of claim 21, wherein the drug is Tenovin-1, Tenovin-6 or Icilin.

23. The method of claim 13, wherein the minor spliceosome inhibitor is mir34A.

24. (canceled)

25. The method of claim 13, wherein the RNA virus is SARS-CoV-2 and the MIGs include PRIM1, POLA2, SBNO1, CRTC3, AP2A2, EXOSC2, EXOSC5, SRP72, HSBP1, NUP210, INTS4, ATP6V1A, UPF1, or a combination thereof.
